(19) United States
(12) Patent Application Publication
(10) Pub. No.: US 2025/0325287 A1
(43) Pub. Date: Oct. 23, 2025

(54) ASPIRATION SYSTEM CONFIGURED FOR ASPIRATION OF VASCULAR DEBRIS FROM THE VASCULATURE

(71) Applicant: Straub Medical AG, Wangs (CH)

(72) Inventors: Tom Lavrijsen, Sargans (CH); Bruno Bahnmueller, Staefa (CH)

(21) Appl. No.: 18/864,312

(22) PCT Filed: May 11, 2022
(86) PCT No.: PCT/EP2022/062748

§ 371 Date: Nov. 8, 2024

(51) Int. Cl. A61B 17/22

(52) U.S. Cl. CPC A61B 17/22; A61B 2017/22079

(57) ABSTRACT

The present disclosure relates to an aspiration system configured for aspiration of vascular debris from the vasculature, the system comprising a catheter comprising at least one or two lumen(s) configured for accommodating vascular debris and transporting vascular debris toward a proximal end of the catheter by way of suction, at least first and second ports in fluid communication with the lumen(s) and for entry of vascular debris from the vasculature into the lumen(s), wherein the first and second ports are located at a distal part of the catheter and are distanced from each other, and the system is configured to facilitate aspiration of vascular debris by agitating vascular debris by providing coordinated pressure pulses, optionally suction pulses, at the first and second ports.

### TECHNICAL FIELD

The present disclosure relates to an aspiration system configured for aspiration of vascular debris from the vasculature. Also, the present disclosure relates to a control unit for controlling aspiration using an aspiration catheter as well as a method for aspiration.

### BACKGROUND

For vascular treatment, in particular removal of occlusions within a blood vessel, aspiration systems are known. In addition to aspiration, further treatments may be added, such as based on thrombectomy or atherectomy.

It is known from US 2015/0342682 A1 that suction provided by a proximal vacuum pump is used to suck in material at a distal end of a catheter. Usually, at the distal end, the catheter has a port serving as an opening for receiving material to be removed from the vasculature.

Pulsing characteristics can be used in connection with aspiration. As such, it is known to utilise a series of constant and/or varying pressure pulses at such port, as this is beneficial in aspirating vascular debris.

In other prior art systems, not only a single port for receiving the vascular debris, but also a second port may be provided.

### SUMMARY

The present disclosure may support improvement of aspiration and/or removal of vascular debris from blood vessels.

According to claim 1, an aspiration system is configured for aspiration of vascular debris from the vasculature, wherein the system comprises a catheter comprising at least one or two lumens configured for accommodating vascular debris and transporting vascular debris toward a proximal end of the catheter by way of suction, and at least first and second ports in fluid communication with the lumen(s) and for entry of vascular debris from the vasculature into the lumens. The first and second ports are located at a distal part of the catheter and are distanced from each other. The system is configured to facilitate aspiration of vascular debris by agitating vascular debris by providing coordinated pressure pulses, optionally suction pulses, at the first and second ports.

Pressure pulses provide discontinuous pressure levels or pressure fluctuations, such that the pressure changes over time. Optionally, the pulses may be periodic. Pressure pulses may be regarded as pressure difference pulses, as the pressure is increased or decreased relative to the ambient pressure. Pressure pulses are applied to the first and second ports in a coordinated way. Optionally, the pressure pulses may be suction pulses. This means that the pressure pulses may be based on negative pressure compared to the ambient pressure by way of suction. However, also the alternative, namely pressure pulses based on increased pressure relative to the ambient pressure, is conceivable.

Coordination of pressure pulses means that the pressure pulses in connection with the first and second ports are not independently from each other or not individually set, but are coordinated relative to each other in that the combination, in particular temporal interaction, of the pulses at the first and second ports supports agitation and, hence, aspiration. Coordination is implemented such that aspiration of vascular debris by agitating the vascular debris by way of the pressure pulses applied at the first and second ports is improved. In particular, it is desired to bring vascular debris into movement (agitation) based on the timing of pressure pulses acting at the first and second ports.

Specifically, the movement of the vascular debris may be between the first and second ports, or more generally speaking, in the opposing directions along which the ports are aligned.

By agitating, i.e. imparting movement to, vascular debris, separation from the vessel wall of the vascular debris is supported, so that the vascular debris becomes loose, possibly also macerated, and can be smoothly sucked in the catheter. Also, it is conceivable that the present disclosure supports aspiration of emboli, i.e. of lose vascular debris, via one of the ports, in that, for example, an embolus may be broken up by way of opposite forces acting at the ports on it.

Generally, as the vascular debris is being sucked in along the lumen(s), suction does not need to be the only force acting on the vascular debris. For example, additional forces originating from a rotating helix inside the lumen may act on the vascular debris.

The ports may be located directly in the catheter tube, or may be indirectly part of the catheter in that the ports are provided in an entity positioned distal to the actual catheter tube, for example.

Optionally, the first and second ports may be positioned along the length direction of the catheter, i.e. the direction of propagation of the pressure wave(s). In other words, the ports may have a different length position in that the ports are located at different distances relative to the distal end of the catheter. As such, the first and second ports may be regarded as proximal and distal ports, respectively. It is, however, not excluded that the ports are at the same longitudinal position

Vascular debris may comprise clot, calcifications, emboli, thrombi, etc. in the vasculature.

Also contemplated is the use of the aspiration system of the present disclosure, and a method for aspirating vascular debris.

Optionally, the system is configured to facilitate reciprocating and/or oscillating movement of vascular debris by way of the pressure pulses and/or due to the arrangement of the locations of the first and second ports. Optionally, the pressure pulses can be alternated between the first and second ports. By initiating a reciprocating movement and/or oscillation of the vascular debris, loosening from the vasculature due to repeated force-exertion may be supported.

In a preferred embodiment, the system further comprises a control unit configured to impart to the catheter pressure pulses at the first and second ports, wherein the pressure pulses at the first port differ from the pressure pulses at the second port, optionally as to the wavelength, further optionally as to the phase.

By way of a selection of the pressure pulses, controlled by a control unit, agitation of the vascular debris can be optimized. The difference in connection with the pressure pulses can be based on the wavelength and/or the phase. As such, by selecting a wavelength for the pressure pulses at the first and second ports, the difference of the pressure pulses at the first and second ports can be chosen accordingly. One way to control the difference is by way of the wavelength, i.e. frequency, of the pressure pulses, and another is the phase of the wave of the pressure pulses relative to each other, i.e. the phase shift between the first and second pulses.

Optionally, the pressure pulses at the first and second ports are periodic and have first and second wavelengths, respectively. The first wavelength is 1.5 to 20, further optionally 2 to 5 times the second wavelength. As the differences in wavelengths are in this range, it is considered that the pulses exert forces onto the vascular debris that result in an appropriate movement and, hence, better removal from the vasculature.

The different wavelengths at the first and second ports may be provided by different vacuum pumps.

As such, different wavelengths may be provided by different pumps, meaning that the first port may be connected to a first pump, and the second port may be connected to a second pump. Specifically in this case, the first and second ports may be positioned at the same length, at different radial positions, i.e. different positions on the circumference of the catheter.

If pressure pulses at the first and second ports are periodic and have the same wavelength, the phase shift between the pressure pulses at the first port relative to the pressure pulses at the second port may substantially fulfil 0.2 to 0.8 times the wavelength. Optionally, the phase shift is 0.2 to 0.6 times the wavelength, further optionally corresponds to one half of the wavelength. In this context, it is noted that it is not required for the pressure pulses to be completely out of phase, but that a non-matching phase may be considered as sufficient, as it already ensures that the pressure difference at one port is higher than the pressure difference at the other report, in one moment in time, and vice versa, so that a periodic change of the prevailing pressure difference leads to a reciprocating movement of the vascular debris in the region of or between the ports.

The phase shift does not need to be constant during a procedure. It is conceivable that the phase shift between the pressure pulses at the first and second ports changes over time. As such, a time-dependent or varying phase shift is generally contemplated. For example, the phase shift may approach or converge towards 0.5 times the wavelength over time during a procedure.

Alternatively or additionally, the wavelength(s) do not need to be constant during a procedure. It is conceivable that the difference in wavelengths (or wavelength ratio) between the pressure pulses at the first and second ports changes over time. As such, a time-dependent or varying wavelength—difference is generally contemplated. For example, the wavelength difference may approach or converge towards 2 over time during a procedure.

In some embodiments, one of the at least two lumens is referred to as first lumen and in fluid connection with the first port, but not in fluid communication with the second port. The other one of the at least two lumens is referred to as second lumen and in fluid communication with the second port, but not in fluid communication with the first port. This may be reflected in embodiments, in which different wavelengths of pressure pulses are provided by different pumps, so that the first pump is connected via the first lumen to the first port, wherein the second pump connects via the second lumen to the second port.

Generally, the first and second lumens may be concentric or in a D-shaped configuration.

Optionally, the catheter comprises a first connecting element for connecting the first lumen to a first pressure system, and a second connecting element for connecting the second lumen to a second pressure system different to the first pressure system. As such, the catheter has separate lumens and allows for connection to a respective pump.

The system may comprise a pressure control unit configured to impart to the catheter pressure pulses at the first and second ports, wherein a phase shift of the pressure pulses applied to the first lumen relative to the pressure pulses applied to the second lumen fulfils 0.2 to 0.8 times the wavelength, optionally 0. 4 to 0.6 times the wavelength, further optionally 0.5 times the wavelength, at the first and second ports. As such, it is the pressure control unit, optionally of the pump, which provides for the phase shift at the different ports.

Optionally, this phase shift may be present at the first connecting element and the second connecting element. This means that the phase shift is applied at the first and second connecting elements, i.e. by the pressure control units at the proximal end of the lumens.

In an embodiment, a first connecting element for connecting the first lumen to a common pressure system, and a second connecting element for connecting the second lumen to the common pressure system are provided. The first and second connecting elements are optionally united and the system further optionally comprises the common pressure system. Accordingly, the catheter may have separate lumens, but the catheter is connected to the same pressure system, e.g. the same pump. As such, the pressure difference between the first and second ports is not based on the pressure system, which is the same pressure system, but the pressure difference is imparted downstream of the pumps.

Alternatively, when the first and second ports are in fluid communication with a common lumen representing the at least one lumen, the system further optionally comprises a common pressure system for supplying the pressure pulses to the common lumen. In such embodiment, a single lumen for connecting the same pressure system with both ports is provided.

In one embodiment, a switch is provided in the common lumen configured for alternatingly switching fluid communication between an open state between the common lumen and the first port, on the one hand, and a substantially closed state between the common lumen and the second port, on the other hand. And vice versa, namely between an open state between the common lumen and the second port, on the one hand, and a substantially closed state between the common lumen and the first port, on the other hand. In other words, the switch switches between a first state characterized by an open state facilitating fluid communication between the common lumen and the first and a substantially closed state in connection with the common lumen and the second port, and a second state characterized by an open state facilitating fluid communication between the common lumen and the second port, and a substantially closed state in connection with the common lumen and the first port. The respective closed state includes a reduction of the fluid communication of at least 50%, optionally at least 30%, or optionally at least 20%, compared to the respective open state. Optionally, a switching interval between the open and closed state fulfils 0.2 to 0.8 times the wavelength, optionally 0.4 to 0.6 times the wavelength, further optionally 0.5 times the wavelength. As such, the switch provides a pressure difference between the first and second ports, so that a prevailing force periodically acts on the vascular debris. The most powerful pressure difference is expected when the switching interval is half of the wavelength.

According to the present disclosure, more than one port is provided. More than two ports are conceivable. One lumen per port may be provided. Alternatively, the ports may share a common lumen.

Optionally, the catheter may comprise further ports in addition to the first and second ports. The further ports are optionally provided at different locations in the direction of propagation of the pressure pulses, optionally along the length direction of the catheter. As per the embodiments as disclosed above for two ports, the additional ports may have a respective lumen, so that optionally, one lumen per port is present in the catheter. Depending on the position relative to each other of the first, second, third, fourth, and so on ports, an appropriate phase shift is to be chosen, so as to support agitation of the vascular debris, in particular reciprocating movement thereof.

As to a control unit of the present disclosure, a control unit may be for controlling aspiration using an aspiration catheter, in particular the aspiration system of the present disclosure. The catheter of the control unit may comprise at least one or two lumens configured for accommodating vascular debris and transporting vascular debris toward a proximal end of the catheter by way of suction, at least first and second ports in fluid communication with the lumens and for entry of vascular debris from the vasculature into the lumens, wherein the first and second ports are located at a distal part of the catheter and are distanced from each other, wherein the control unit is configured to control coordinated application of pressure pulses at the first and second ports such that vascular debris is agitated.

Optionally, the control unit is configured to facilitate reciprocating movement of vascular debris by way of the pressure pulses, optionally by alternating pressure pulses between the first and second ports.

Optionally, the control unit is configured to control the catheter pressure pulses at the first and second ports such that the pressure pulses at the first port differ from the pressure pulses at the second port, optionally as to the wavelength, further optionally as to the phase.

Optionally, the pressure pulses at the first and second ports are periodic and have first and second wavelengths, respectively, wherein the first wavelength is 1.5 to 20, optionally 2 to 5 times the second wavelength.

The catheter may comprise one of the at least two lumens referred to as first lumen and in fluid communication with the first port, but not in fluid communication with the second port. The other one of the at least two lumens is referred to as second lumen and in fluid communication with the second port, but not in fluid communication with the first port. The control unit is configured to control the pressure pulses at the first and second ports such that a phase shift between the pressure pulses applied to the first lumen relative to the pressure pulses applied to the second lumen fulfils 0.2 to 0.8 times the wavelength, optionally 0.4 to 0.6 times the wavelength, further optionally 0.5 times the wavelength at the first and second ports.

Optionally, the catheter may comprise a first connecting element for connecting the first lumen to the first pressure system, and a second connecting lumen for connecting the second lumen to a second pressure system other than the first pressure system, wherein the phase shift is 0.2 to 0.8 times the wavelength, optionally 0.4 to 0.6 times the wavelength, further optionally 0.5 times the wavelength between the pressure pulses at the connecting element and the pressure pulses at the second connecting element.

The present disclosure also relates to the method of aspirating vascular debris from the vascular system.

### BRIEF DESCRIPTION OF THE DRAWINGS

FIG. 1a shows a cross-section of a vessel with a catheter positioned within the vessel.

FIG. 1b shows wavelengths according to the disclosure.

FIG. 2a shows a catheter having separate lumens.

FIG. 2b shows a proximal part of a catheter having two separate lumens and two pressure systems.

FIG. 3 shows a catheter with a pressure system and a control unit, wherein the catheter has a switch.

### DETAILED DESCRIPTION

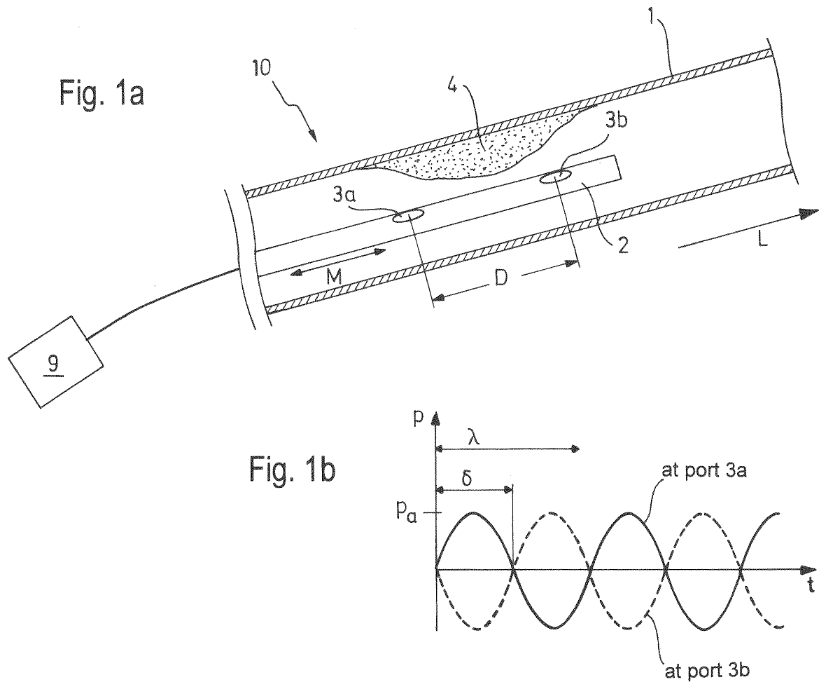

The present disclosure relates to an aspiration system 10 configured for aspiration of vascular debris 4 from the vasculature, i.e. the vessel walls 1. The system 10 comprises a catheter 2 comprising at least two lumens 5. The catheter 2 extends in the longitudinal direction L of the catheter. In FIG. 1a, first 3a and second 3b ports according to a first embodiment are shown. The lumen(s) 5 is/are configured for accommodating vascular debris 4 and transporting vascular debris 4 towards a proximal end of the catheter 2 by way of suction. At least the first 3a and second 3b ports are in fluid communication with the lumen 5 and allow for entry of vascular debris 4 from the vasculature 1 into the lumens 5.

The first 3a and second 3b ports are located at a distal part of the catheter 2 and are distanced from each other by a distance D. The system 10 is configured to facilitate aspiration of vascular debris 4 by agitating vascular debris 4 by providing coordinated pressure pulses at the first 3a and second 3b ports. The coordinated pressure pulses between the ports 3a and 3b act on the debris 4, which is, in the state shown in FIG. 1a, still connected to the vessel wall 1. Upon suction by way of the ports 3a and 3b and the coordinated pulses, the vascular debris 4 is moved and, due to the change of direction of the suction due to the coordinated pressure pulses at the ports 3a and 3b, a movement of the vascular debris along the direction of the arrow M is initiated. By way of this movement, loosening and separation of the vascular debris 4 from the vessel wall 1 is supported. In a subsequent state, not shown in the drawings, the vascular debris 4 would, as loosened emboli, be present directly between the ports 3a and 3b and would be sucked in via one of these port, or by both ports if the emboli is broken up, for example.

The system 10 allows for reciprocating movement, i.e. oscillating movement in the opposite directions along the arrow M of vascular debris 4 by way of the pressure pulses. Between the first 3a and second 3b ports, the pressure pulses may alternate, already because of the location of the first 3a and second 3b ports.

As shown in FIG. 1a, the system 10 further comprises a control unit 9 configured to impart to the catheter 2 pressure pulses at the first 3a and second 3b ports. The pressure pulses at the first port 3a differ from the pressure pulses at the second port 3b. In particular, the difference may be the wavelength and/or the phase.

The pressure pulses may be periodic. The pressure pulses may have first and second wavelengths λ, wherein the first wavelength may differ from the second wavelength. For example, the first wavelength may be 1.5 to 20, or to 2 to 5 times the second wavelength.

FIG. 1b reflects pressure pulses having the same wavelength λ. In this example, the wave is a sine wave. The phase shift δ between the pressure pulses at the first port 3a and the second port 3b is half of the wavelength λ. As such, the pressure pulses are exactly opposite to each other, which is expected to result in the largest pressure difference between the ports 3a and 3b in one moment in time and, as such, to impart the maximum movement onto the vascular debris 4. The phase shift δ may be constant during a procedure or may change over time, i.e. the phase shift may vary. Alternatively or additionally, it is conceivable that the difference in wavelength, i.e. the difference between the wavelength at the first port 3a and the wavelength at the second port 3b, varies over time. For example, while the wavelength at first port 3a may be constant, the wavelength at the second port 3b may vary during a procedure, or vice versa, or both wavelengths may vary over time.

FIG. 1b indicates an amplitude of the wave corresponding to the maximum pulse pressure difference. The pressure pₐ is considered as the pressure of the ambient air. It is considered that the absolute amplitude of the pressure is of less relevance in connection with the present disclosure. The amplitude of the pressure pulses may differ between the first and second ports, i.e. between the first and second pressure pulses, or may be the same.

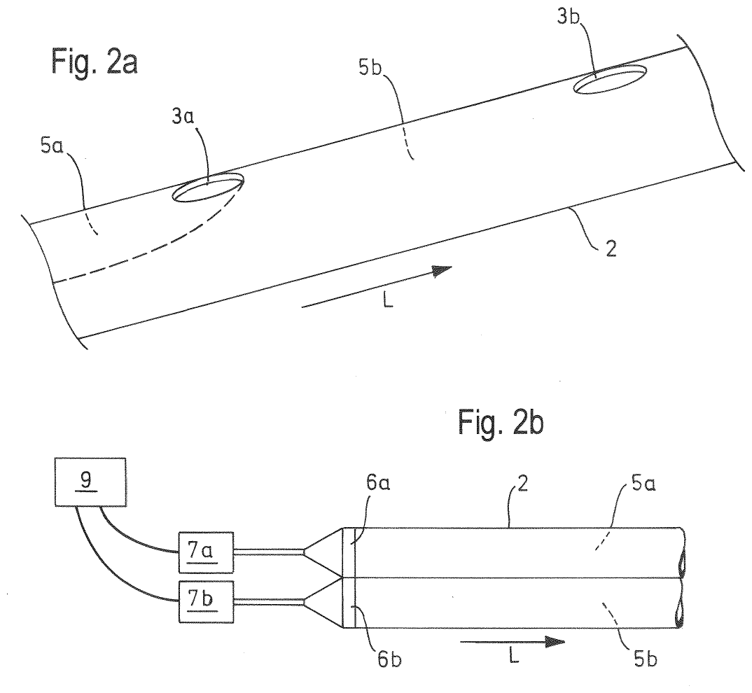

FIG. 2a shows a second embodiment having a catheter 2 having a first port 3a in fluid communication with the first lumen 5a. The first lumen 5a is not in fluid communication with the second port 3b. The second lumen 5b is in fluid communication with the second port 3b, but not in fluid communication with the first port 3a. The pressure pulses may be coordinated based on the distance D between the ports 3a and 3b in the length direction D. Alternatively or additionally, the following features may be realized:

In FIG. 2b, the catheter 2 further comprises a first connecting element 6a for connecting the first lumen 5a to a first pressure system 7a, and a second connecting element 6b for connecting the second lumen 5b to a second pressure system 7b other than the first pressure system 7a. A first connecting element 6a and a second connecting element 6b are provided. Hence, as shown in FIG. 2b, a first subsystem is formed by the first port 3a, the first lumen 5a and the first pressure system 7a, whilst a second subsystem is formed by the second port 3b, the second lumen 5b, and the second pressure system 7b.

A control unit 9 is configured to impart to the catheter 2 pressure pulses for the first 3a and second 3b ports. A phase shift between the pressure pulses applied to the first lumen 5a relative to the pressure pulses applied to the second lumen 5b is ideally half of the wavelength at the first 3a second 3b ports. The phase shift at the first 3a and second 3b ports may correspond to the pressure difference at the first connecting element 6a and the second connecting element 6b already. While FIG. 2b shows two different pressure system 7a and 7b, it is conceivable that a common pressure system 7 (not shown in the drawings) is used. As such, a first connecting element 6a for connecting the first lumen 5a to the common pressure system 7, and a second connecting element 6b for connecting the second lumen 5b to the common pressure system 7 may be provided. It is conceivable that the first and second connecting elements 6a and 6b are united and that the system 10 further comprises the common pressure system 7.

While the schematic drawing of FIG. 2b shows two lumens 5a and 5b positioned next to each other, it is contemplated to position the lumens 5a and 5b differently than next to each other. For example, the lumens 5a and 5b may be concentric or in a D-shaped arrangement (i.e. one lumen having a crescent shape and the other lumen having a corresponding shape, such as a circular shape substantially surrounded by the crescent).

At the proximal end of the catheter, a handle (not shown in the drawings) may be provided. The handle may be reusable, while only the catheter 2 is disposable. Alternatively, the handle may be disposable as well and may even include a pressure system, such as a pump, which is disposable.

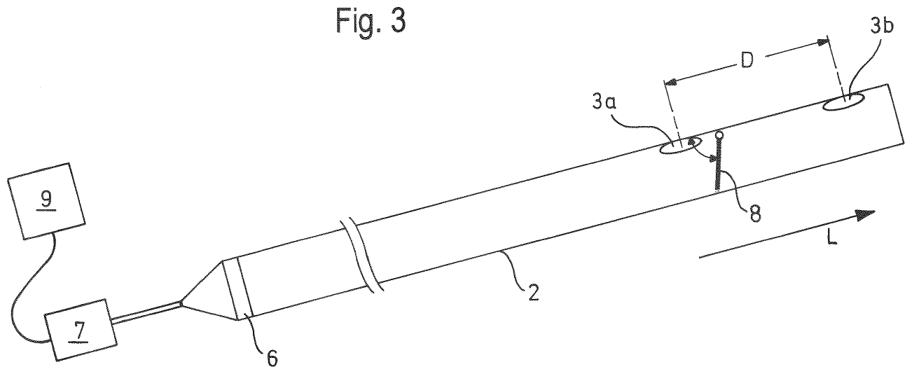

In a third embodiment, as shown in FIG. 3, a common lumen 5, a common connecting element 6, as well as a common pressure system 7 are provided. Within the catheter 2, towards the distal end, optionally downstream the first port 3a, but upstream the second port 3b, a switch 8 is provided. In an embodiment, the switch 8 may be a flap, which may be pivoted such that, in an open state, the lumen 5 connects with the second port 3b, whilst the first port 3a is closed, and a substantially closed state in connection with the first port 3a is achieved. The switching interval t between the open and closed state may, ideally, be half of the wavelength of the pressure pulses imparted by the pressure system 7. As such, (positive or negative) pressure is applied only via the first 3a or second 3b port. As indicated in FIG. 3, the distance between the first 3a and second 3b port is D and may correspond to approximately half of the wavelength. In this case, no switch 8 may be needed, when the distance D corresponds to half of the wavelength, for example.

Specifically, the distance D refers to the direction of propagation of the pressure pulses, in particular the length direction L of the catheter. The catheter is configured to provide the pressure pulses at the first 3a and second 3b ports, which pressure pulses are periodic and have the same wavelength.

Although the drawings show embodiments having first 3a and second 3b ports, also additional ports 3 may be provided. Further ports may be in between the first 3a and second 3b ports. Optionally, the additional ports are provided at different locations in the direction of propagation of the pressure pulses, in particular the length direction L of the catheter.

The present invention also refers to a control unit 9, as shown in the drawings, the control unit 9 configured to control coordinated application of pressure pulses at the first 3a and second 3b ports (or further ports) such that vascular debris is being agitated.

### LIST OF REFERENCE SIGNS

1 vessel wall
2 catheter
3 port
4 vascular debris
5 lumen
6 connecting element
7 pressure system (pump)
8 switch
9 control unit
10 aspiration system
L longitudinal/length direction of catheter
M directions of movement
D distance

1. An aspiration system configured for aspiration of vascular debris from the vasculature, the system comprising a catheter comprising:
at least one or two lumen(s) configured for accommodating vascular debris and transporting vascular debris toward a proximal end of the catheter by way of suction,
at least first and second ports in fluid communication with the lumen(s) and for entry of vascular debris from the vasculature into the lumen(s), wherein
the first and second ports are located at a distal part of the catheter and are distanced from each other, and
the system is configured to facilitate aspiration of vascular debris by agitating vascular debris by providing coordinated pressure pulses at the first and second ports.

2. The system of claim 1, wherein the system is configured to facilitate reciprocating and/or oscillating movement of vascular debris by way of the pressure pulses and/or due to the positions of the locations of the first and second ports.

3. The system of claim 1, further comprising a control unit configured to impart to the catheter pressure pulses at the first and second ports, wherein the pressure pulses at the first port differ from the pressure pulses at the second port.

4. The system of claim 3, wherein the pressure pulses at the first and second ports are periodic and have first and second wavelengths, respectively, wherein the first wavelength is 1.5 to 20.

5. The system of claim 1, wherein the system is configured to provide pressure pulses at the first and second ports, which pressure pulses are periodic and have the same wavelength λ, and a phase shift θ between the pressure pulses at the first port relative to the pressure pulses at the second port substantially fulfils θ=(0.2 to 0.8)*λ, θ=(0.4 to 0.6)*λ, or θ=0.5*λ.

6. The system of claim 1, wherein:
the at least one or two lumens is at least two lumens,
one of the at least two lumens is a first lumen and in fluid communication with the first port, but not the second port, and
the other one of the at least two lumens is a second lumen and in fluid communication with the second port, but not with the first port.

7. The system of claim 6, wherein the catheter further comprises a first connecting element for connecting the first lumen to a first pressure system and a second connecting element for connecting the second lumen to a second pressure system different to the first pressure system.

8. The system of claim 6, further comprising a control unit configured to impart to the catheter pressure pulses at the first and second ports, wherein a phase shift θ between the pressure pulses applied to the first lumen relative to the pressure pulses applied to the second lumen fulfils θ=(0.2 to 0.8)*λ, θ=(0.4 to 0.6)*λ, or θ=0.5*λ at the first and second ports.

9. System of claim 7, wherein the phase shift θ fulfils θ=(0.2 to 0.8)* )λ, θ=(0.4 to 0.6)*λ, or θ=0.5*λ between the pressure pulses at the first connecting element and the pressure pulses at the second connecting element.

10. The system of claim 1, wherein the catheter further comprises
a first connecting element for connecting a first lumen to a common pressure system, and a second connecting element for connecting a second lumen to said common pressure system.

11. The system of claim 1, wherein the first and second ports are in fluid communication with a common lumen.

12. The system of claim 11, wherein the catheter comprises
a switch in the common lumen, the catheter configured for alternatingly switching between a first state characterized by an open state facilitating fluid communication between the common lumen and the first port, and a substantially closed state in connection with the common lumen and the second port, and a second state characterized by an open state facilitating fluid communication between the common lumen and the second port, and a substantially closed state in connection with the common lumen and the first port, wherein the respective closed state includes a reduction of the fluid communication of at least 50%.

13. The system of claim 1, wherein a distance D between the first and second ports fulfils D=(n+0.2 to 0.8)* λ, D=(n+0.4 to 0.6)* λ, or D=(n+0.5)*λ, n=0, 1, 2, 3 . . . , wherein the distance D refers to the direction of propagation of the pressure pulses.

14. The system of claim 1, wherein the catheter comprises further ports in addition to the first and second ports.

15. A control unit for controlling aspiration using an aspiration catheter, the catheter comprising:
at least one or two lumen(s) configured for accommodating vascular debris and transporting vascular debris towards a proximal end of the catheter by way of a pressure difference, and
at least first and second ports in fluid communication with the lumen(s) and for entry of vascular debris from the vasculature into the lumen(s), wherein
the first and second ports are located at a distal part of the catheter and are distanced from each other, wherein
the control unit is configured to control coordinated application of pressure pulses at the first and second ports such that vascular debris is agitated.

16. The control unit of claim 15, wherein the control unit is configured to facilitate reciprocating movement of vascular debris by way of the pressure pulses.

17. The control unit of claim 15, wherein the control unit is configured to control the catheter pressure pulses at the first and second ports such that the pressure pulses at the first port differ from the pressure pulses at the second port.

18. The control unit of claim 17, wherein the pressure pulses at the first and second ports are periodic and have first and second wavelengths, respectively, wherein the first wavelength is 1.5 to 20, optionally 2 to 5 times the second wavelength.

19. The control unit of claim 15, wherein:
the catheter comprises a first lumen in fluid communication with the first port, but not the second port, and a second lumen in fluid communication with the second port, but not with the first port, and
the control unit configured to control the pressure pulses at the first and second ports such that a phase shift θ between the pressure pulses applied to the first lumen relative to the pressure pulses applied to the second lumen fulfils θ=(0.2 to 0.8)*λ, θ=(0.4 to 0.6)*λ, or θ=0.5*λ at the first and second ports.

20. The control unit of claim 19, wherein:
the catheter comprises a first connecting element for connecting the first lumen to a first pressure system and a second connecting element for connecting the second lumen to a second pressure system different to the first pressure system, and
wherein the phase shift θ fulfils θ=(0.2 to 0.8)*λ, θ=(0.4 to 0.6)*λ, or θ=0.5*λ, between the pressure pulses at the first connecting element and the pressure pulses at the second connecting element.
